(19) United States
(12) Patent Application Publication
(10) Pub. No.: US 2024/0428924 A1
(43) Pub. Date: Dec. 26, 2024

(54) SYSTEMS AND METHODS FOR PREDICTING CRISIS SCENARIOS AND INITIATING SUICIDE INTERVENTION

(71) Applicant: Wellness Empowered, LLC, San Antonio, TX (US)

(72) Inventors: Gerard Berlanga, San Antonio, TX (US); Antonio Santana, San Antonio, TX (US); Carl Burnside, Cedar Creek, TX (US)

(73) Assignee: Wellness Empowered, LLC, San Antonio, TX (US)

(21) Appl. No.: 18/749,124

(22) Filed: Jun. 20, 2024

Related U.S. Application Data
(60) Provisional application No. 63/522,072, filed on Jun. 20, 2023.

(51) Int. Cl. G16H 20/70; G06Q 50/26

(52) U.S. Cl. CPC G16H 20/70; G06Q 50/265

(57) ABSTRACT

A system and method for predicting crisis scenarios and initiating suicide intervention is disclosed. The system and method provide proactive suicide prevention by predicting crises based on user data analysis and initiating appropriate interventions. Artificial intelligence and machine learning techniques are employed to enhance crisis prediction capabilities. The system comprises a user device interface for obtaining user data, an intake and setup engine for assigning risk status to user profiles, an action plan engine for managing action plans, a user data analysis engine for analyzing user data and predicting crisis scenarios, and a crisis intervention engine for initiating intervention protocols. The method involves obtaining user data, assigning risk status, analyzing the data to identify patterns and predict crises, generating user feedback, and initiating intervention protocols based on the assigned risk status.

### BACKGROUND

Existing approaches to suicide prevention are generally reactive in that they are implemented after the occurrence of a circumstance or situation where an individual was at risk of committing suicide. These approaches generally rely on lagging measures or indicators such as self reported thoughts, feelings, emotions, lifestyle choices etc. discussed with a medical professional after the occurrence of the circumstance(s) associated with a potential for self-harm or suicide, or attempted self-harm or suicide. While this approach allows for establishing measures for trying to avoid future dangerous circumstances, there are limited observations or monitoring of patient well-being wherein an effective intervention can be implemented. These approaches are not proactive, not predictive and generally fail to allow for in the moment intervention where prevention of self harm or suicidal actions can be implemented.

Moreover, this approach to treating at-risk patients relies on patient compliance in properly recording or journaling mood and lifestyle information over time for later review by or with a clinician (e.g. therapist) and generally lacks additional data (e.g. no HR, pulse, sleep, etc.) which could be beneficial in identify at-risk circumstances. This approach also requires an individual to be able to recall something that may have happened several weeks ago in which case the details around that situation may not be easily remembered or recalled by a patient making it difficult to discuss the situation and making it difficult for a therapist to address it and provide an appropriate treatment or change in treatment.

Furthermore, conventional approaches generally lack the ability to provide ongoing real-time feedback to users as to how certain lifestyle choices are directly impacting their mood and/or mental health. In addition, the current treatment model fails to timely provide information which would allow a patient to self-learn and self-correct certain behaviors having a negative impact on their mood as the current treatment model is reactive in nature. Moreover, the current approaches place the burden on the patient in recognizing circumstances

### SUMMARY

The present invention overcomes these limitations by creating a user/patient monitoring and suicide intervention system which proactively predicts scenarios indicative of suicide risk and initiates intervention protocols when such are identified. Furthermore, the present invention provides users with real-time feedback regarding lifestyle actions and mental health status allowing users to self-learn and modify behaviors prior to reaching the extreme circumstances leading to suicidal circumstances. The present invention enables therapists to have additional insight into patient lifestyle characteristics via the ongoing data collection and analysis, alerts when crisis scenarios are detected thereby allowing more immediate intervention by therapists and ultimately improving patient treatment and prevention of suicide.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a system for patient interaction, monitoring and suicide intervention in accordance with an exemplary embodiment of the invention.

FIG. 2 illustrates a system for user monitoring and suicide intervention in accordance with an exemplary embodiment of the present invention.

FIG. 3 illustrates an exemplary process for user monitoring and suicide intervention according to one embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

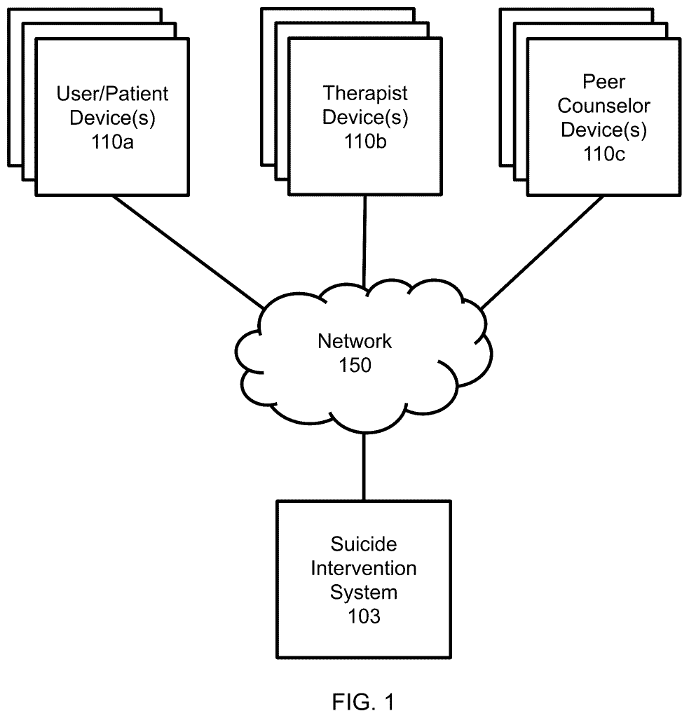

FIG. 1 illustrates an exemplary embodiment of a system for patient interaction, monitoring and suicide intervention according to one embodiment. The system includes user/patient device(s) 110a, therapist device(s) 110b, peer counselor device(s) 110c, suicide intervention system 103, and a network 150 over which the various systems communicate and interact. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

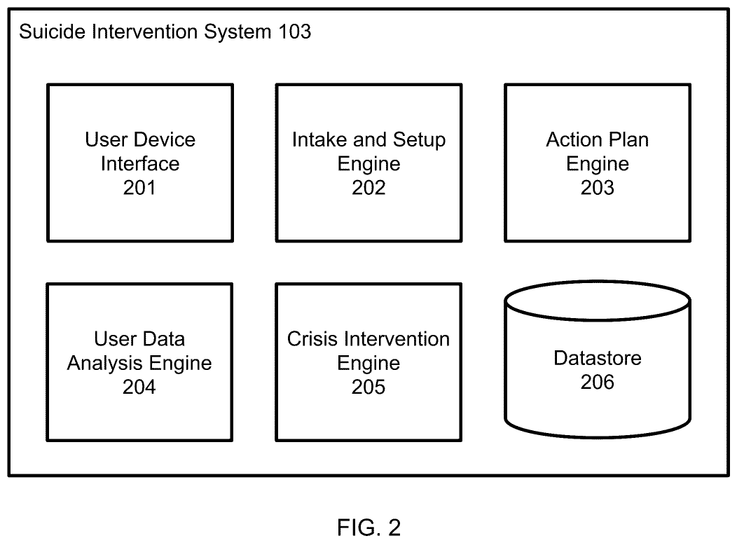

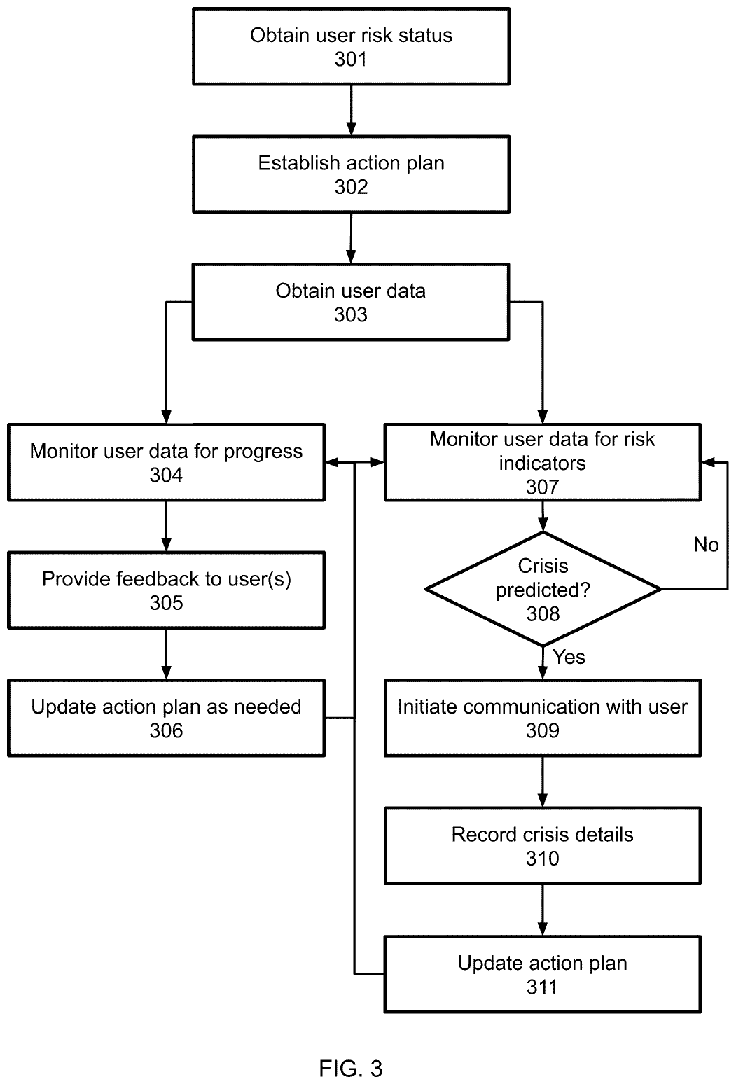

Suicide intervention system 103 is operable to obtain input from at least one user device (including patient devices 110a, therapist devices 110b, and peer counselor devices 110c) and implement interventional processes to reduce the likelihood of suicide of the patient. Suicide intervention system 103 may obtain, from user/patient devices, at least one of data associated with lifestyle activities (e.g. sleep, exercise, heart rate), data associated with mood (e.g. user provided mood information), and journal data (e.g. user provided journaling of mood and associated activities or circumstances). Suicide intervention system 103 may analyze the obtained data in order to provide users with feedback regarding progress and/or trends associated with mental health status such that patient's are provided with an opportunity for self-learning and lifestyle adjustment. Suicide intervention system 103 may analyze the obtained data in order to predict crisis circumstances or scenarios and implement suicide intervention measures. Suicide intervention system may obtain data from therapist devices 110b such as intake and setup information including suicide risk assessment information, action plan information, and communication information between therapist and patient as well as initiate and/or alert individuals of the need for communication with a user/patient. Additional details of the suicide intervention system 103 are discussed below in association with FIGS. 2-3.

User device(s) 110 (including user/patient devices 110a, therapist devices 110b, and peer counselor devices 110c) include, generally, a computer or computing device including functionality for communicating (e.g., remotely) over a network 150. User device(s) 110 are operable to obtain data from users and provide data to other system components via network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, smart watch or other wearable computing device, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

FIG. 2 illustrates an exemplary embodiment of the suicide intervention system 103. The suicide intervention system 103 includes user device interface 201, intake and setup engine 202, action plan engine 203, user data analysis engine 204, crisis intervention engine 205, and datastore 206. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

User device interface 201 is operable to communicate with at least one of user/patient devices, therapist devices, and peer support counselor devices and obtain data from said devices. User device interface 201 may obtain at least one of health and/or physiology data (e.g. sleep data, heart rate data, etc.), mood data, and journal data from at least one user/patient device. User device interface 201 may obtain action plan information from therapist user devices. User device interface 201 may provide communication to user devices and/or obtain communications from user devices during a predicted crisis scenario.

Intake and setup engine 202 is operable to at least one of manage risk assessment status and assign risk status indicators to user/patient profiles. Intake and setup engine 202 may use therapist assessment information and/or risk indication input to establish and/or associate a user profile with an appropriate risk status indication such that appropriate steps can be taken if and when a crisis scenario is predicted.

Action plan engine 203 is operable to at least one of obtain initial action plan data, obtain updated action plan data, generate alerts associated with user behavior characteristics which deviate from an action plan, and generate notifications associated with action plan progress (e.g. goals/objectives achieved. For example, action plan engine may generate alerts and/or notifications associated with action plan deviations and/or achievements as determined by user data analysis engine 204 (discussed below).

User data analysis engine 204 is operable to at least one of analyze user/patient data to identify patterns and/or trends in user data, identify shifts above/below thresholds, and use artificial intelligence/machine learning (AI/ML) in the analysis of user data. In one aspect, user data analysis engine 204 generates feedback data to be provided to a user for purposes of self-learning and behavior regulation and/or adjustment. In one aspect, user data analysis engine 204 predicts crisis circumstances based on the analyzed user data. In one aspect, user data analysis engine 204 employs AI/ML in identifying patterns, trends, and/or shifts. In one aspect, user data analysis engine 204 uses AI/ML in predicting crisis, such as when the patterns, trends and/or shifts are indicative of a crisis scenario. Additional details of the user data analysis is described below in association with FIG. 3.

Crisis intervention engine 205 is operable to initiate crisis intervention protocols when a crisis scenario is determined by user data analysis engine 204. Crisis intervention engine 205 may implement an intervention protocol as appropriate for a user's assigned risk status. For example, a first protocol may be implemented for lower risk users while a second protocol may be implemented for higher risk users. Crisis intervention engine 205 may trigger communication with at least one of a user/patient and a therapist upon determination of a predicted crisis. The communication may be determined as a function of risk status assessment. For example, lower risk may be associated with a messaging prompt or text message while higher risk may be associated with more urgency and include establishing phone communication with the user/patient within a certain time frame. In circumstances where communication with a user/patient cannot be established (within the established time frame), the crisis intervention engine may trigger safety plan actions such as notifying emergency contacts and/or emergency medical services. Additional details of the crisis intervention processes is described below in association with FIG. 3.

Datastore 206 is operable to store data associated with the various system components described herein. Datastore 206 may store data associated with the general functions of the suicide intervention system 103. Datastore 206 may be used to aggregate data over time for use in at least one of research, education, and training AI/ML algorithms.

FIG. 3 illustrates an exemplary process for predicting crisis circumstances and initiating intervention according to one embodiment of the invention. The process comprises obtaining user risk status 301, establishing an action plan 302, obtaining user data 303, monitoring user data for progress 304, providing feedback to users 305, updating action plan 306, monitoring user data for risk indicators 307, determining whether predictive crisis criteria have been met 308, initiating communication with the user 309, recording crisis details 310, and updating action plan 311. The process steps described herein may be performed in association with a system such as that described in FIG. 1 and/or FIG. 2 above or in association with a different system. The process may comprise additional steps, fewer steps, and/or a different order of steps without departing from the scope of the invention as would be apparent to one of ordinary skill in the art.

At step 301, the process comprises obtaining user risk status. The user may be a patient undergoing treatment related to mental health concerns (e.g. mental health issues associated with suicide risk). Risk status may be established via an assessment of the user by a therapist (e.g. via an intake appointment or consultation). Obtaining risk status may comprise assigning a risk status indicator to a profile associated with the user/patient. Risk status may comprise a plurality of risk tiers indicative of an expected risk of suicidal action by the user. For example, risk tiers may comprise low risk, medium or average risk, and high risk. These are merely exemplary and fewer or a greater number of risk tiers may be used without departing from the scope of the invention.

At step 302, the process may comprise obtaining action plan information. Action plan information may comprise recommendations, such as, but not limited to, lifestyle changes, behaviors, patterns, factors, and medications associated with improving and/or maintaining mental health status. The action plan may comprise hindrances, triggers and/or other recommended avoidances which may be associated with diminishing mental health status. Recommendations may comprise, but are not limited to, medication, sleep recommendations, diet recommendations, exercise recommendations, frequency of therapy/counseling and/or attending support groups, identification of help and support resources, goals/objectives, etc.

At step 303, the process comprises obtaining user data. User data may comprise at least one of sleep, mood, and journal data. The data may be obtained at recurring intervals such that changes over time can be identified. For example, data may be obtained multiple times per day, on a daily basis, weekly basis, etc. The process may proceed to steps 304 and/or 307 at any point in time using obtained data and subsequently return to 303 to continue gathering additional data for use in further monitoring and analysis. The data may be obtained from at least one of a wearable device worn by the user and a user device as described in FIG. 1 above (e.g. smartphone, tablet, computing device, etc.). For example, sleep data may be obtained from a smartwatch and/or smartphone while mood and journal data may be obtained from a smartphone, tablet or other computing device operable to provide the relevant information. In one aspect, physiology data may be obtained from at least one user device (e.g. smartwatch, smartphone, etc.) and may be used in determining sleep and/or mood and changes associated therewith. For example, changes in heart rate and/or pulse may serve as an indication of sleep status and/or changes in mood. Mood data may be obtained from one or more questions presented to the user throughout a day and repeated over time (e.g. daily, weekly, etc.). Mood data may be obtained via prompts on the user device at periodic intervals and may comprise providing at least one of a written response and a selection from a plurality of response options (e.g. a list of common moods). Journal data may comprise at least one of written data, audio data, and voice to text data obtained from a user via a user device(s). Journal data may comprise data associated with circumstances around various events and/or moods or mood changes which occur throughout the course of a day and may be repeatedly obtained on a recurring basis (e.g. daily, weekly, etc.).

At step 304, the process comprises monitoring the obtained user data for progress. In one aspect, the obtained user data may be analyzed to identify trends and/or changes in the obtained data over time. The trends and/or changes may be related to aspects of the action plan associated with the user. The trends and/or changes may be computed relative to a baseline or typical status for the user (e.g. above or below a norm for the user) and/or may reflect periodic changes (e.g. day to day changes, week to week changes, etc.).

At step 305, the process comprises providing feedback to the user regarding progress status. Feedback may comprise notifications presented to the user of positive trends and/or alerts for negative trends. In this way a user is provided information about their progress, what aspects are moving in a direction favorable to mental health, what aspects are moving in a direction unfavorable to mental health status, ultimately allowing the user to self-learn and adjust their lifestyle accordingly.

At step 306, the process comprises updating the action plan as needed. Updating the action plan may comprise making changes to the action plan if an existing action plan is failing to provide the intended results. Updating the action plan may comprise making changes to the action plan if the user has progressed such that aspects of the action plan are no longer relevant (e.g. certain goals/objectives achieved).

At step 307, the process comprises monitoring user data for risk indicators. Risk indicators may comprise behavioral characteristics which exceed and/or fail to meet certain criteria or thresholds. For example, a risk indication may be triggered if sleep duration falls below a threshold level for a plurality of consecutive days, this may trigger a risk indication. As another example, a risk indication may be triggered if total sleep over a plurality of days falls below a threshold level. Another risk indicator trigger may comprise identifying a sudden change in mood from one data point to another (e.g. mood swing within the same day, from day to day, etc.). In one aspect, monitoring user data for risk indicators comprises use of artificial intelligence (AI) and/or machine learning to identify risk indicators. AI algorithms may be trained using aggregated data across a plurality of users and/or individuals with (or without) mental health issues. AI algorithms may be trained using user specific data such that the algorithm is unique to a given individual. In one aspect, monitoring user data for risk indicators may comprise analyzing at least one of mood data and journal data using AI language processing algorithms such as natural language processing (NLP) models and large language models.

At step 308, the process comprises determining if a crisis situation is predicted based on the monitoring performed in step 307. If no crisis is predicted, the process continues monitoring user data for risk indicators as new data is continually obtained at step 303. If a crisis is predicted, the process triggers an intervention protocol as described below.

At step 309, the process comprises initiating communication with user when a crisis scenario is predicted. Communication with a user may comprise at least one means of communication for contacting the user. Communication with the user may comprise a tiered response based on risk status (from step 301). For example, lower risk status may be associated with at least one of a prompt via an application on a user device associated with the user and a text message sent to the user. Higher risk status may be associated with a direct phone call to the user. In one aspect, the process comprises alerting a therapist and/or peer support counselor associated with the user that a phone call to the user is required. Alerting the therapist and/or peer counselor may comprise at least one of a prompt on a user device associated with the therapist and/or peer counselor, a text message to the therapist and/or peer counselor, and a phone call to the therapist and/or peer counselor. Subsequently, the phone call to the user may be initiated by the therapist and/or peer support counselor. In scenarios where the therapist and/or peer support counselor are unable to make the phone call to the user, alternate support personnel (e.g. crisis intervention hotline personnel) may be alerted of the need to call the user.

The communication with the user may be associated with a maximum allowable time period for establishing communication (e.g. within 5 minutes, 10 minutes, etc.) the duration of which may be based on the user risk status. In one aspect, the communication to the user may require a response from the user (e.g. acknowledging a prompt, answering a prompt question, replying to a text message, answering the phone call, etc.). If no response is received the process may trigger the activation of a safety plan. The safety plan may comprise at least one of alerting emergency contacts, alerting emergency medical services, obtaining geolocation information associated with at least one user device, and providing the geolocation information to at least one entity associated with the safety plan.

At step 310, the process comprises recording crisis details. The details may be stored in a database for later retrieval and review. Recording crisis details may comprise identifying (e.g. flagging, highlighting or otherwise associating) the relevant user data associated with the crisis. The recorded user data may serve as training data for use in training predictive crisis models. The recorded user data may be provided to a therapist for use in applying or adjusting treatment accordingly.

At step 311, the process comprises updating the action plan. If a crisis scenario was predicted and was not associated with a false positive prediction, this may be an indication that the previously established action plan requires attention. Therefore, the action plan may be updated to address the issues which likely led to the crisis scenario in an effort to prevent future scenarios from arising. Any of the above mentioned action plan aspects may be adjusted and/or new action plan items added as would be appropriate for the given user/patient and the corresponding situation.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

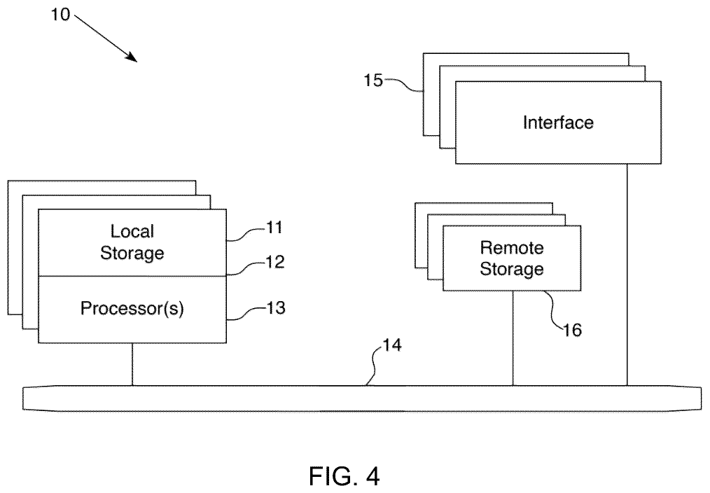

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components or the like may be and/or comprise hardware and/or software as described herein. For example, the suicide intervention system 103 and subcomponents thereof may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

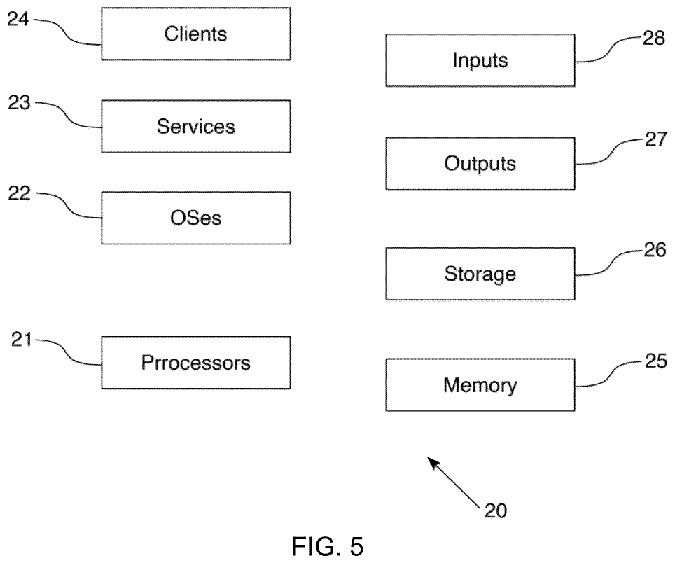

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

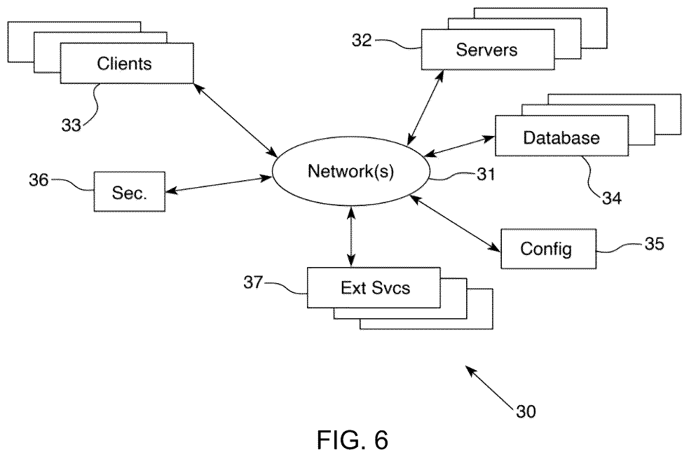

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

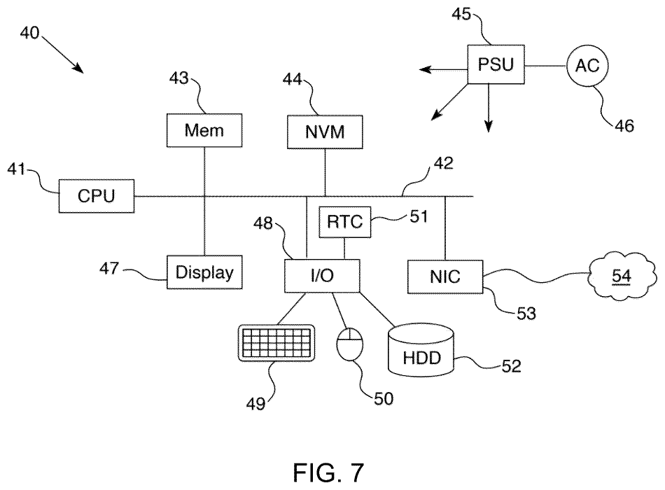

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A system for predicting crisis scenarios and initiating suicide intervention, comprising:
a user device interface configured to communicate with at least one user device and obtain user data from the at least one user device, the user data comprising at least one of health data, physiology data, mood data, and journal data;
an intake and setup engine configured to assign a risk status to a user profile associated with the at least one user device;
an action plan engine configured to obtain action plan data associated with the user profile;
a user data analysis engine configured to:
analyze the user data to identify at least one of patterns, trends and shifts in the user data;
generate feedback data to be provided to the at least one user device based on the analysis; and
predict a crisis scenario based on the analysis; and
a crisis intervention engine configured to initiate a crisis intervention protocol when the crisis scenario is predicted, the crisis intervention protocol determined based on the assigned risk status.

2. The system of claim 1, wherein the at least one user device comprises at least one of a patient user device, a therapist user device, and a peer support counselor user device.

3. The system of claim 1, wherein the user data analysis engine is further configured to employ artificial intelligence and machine learning algorithms in analyzing the user data and predicting the crisis scenario.

4. The system of claim 1, wherein initiating the crisis intervention protocol comprises:
determining a communication protocol based on the assigned risk status;
initiating communication with the at least one user device according to the communication protocol;
activating a safety plan if a response is not received from the at least one user device within a predetermined time period.

5. The system of claim 4, wherein the communication protocol for a low risk status comprises at least one of generating a prompt via an application on the at least one user device and sending a text message to the at least one user device.

6. The system of claim 4, wherein the communication protocol for a high risk status comprises initiating a phone call to the at least one user device.

7. The system of claim 6, further comprising alerting at least one of a therapist user device and a peer support counselor user device of a requirement to initiate the phone call to the at least one user device.

8. The system of claim 4, wherein activating the safety plan comprises at least one of:
alerting an emergency contact;
alerting emergency medical services;
obtaining geolocation information associated with the at least one user device; and
providing the geolocation information to at least one entity associated with the safety plan.

9. The system of claim 1, further comprising recording crisis details when the crisis scenario is predicted, the crisis details comprising user data associated with the crisis scenario.

10. The system of claim 9, further comprising updating the action plan based on the recorded crisis details.

11. A method for predicting crisis scenarios and initiating suicide intervention, comprising:
obtaining, via a user device interface, user data from at least one user device, the user data comprising at least one of health data, physiology data, mood data, and journal data;
assigning, via an intake and setup engine, a risk status to a user profile associated with the at least one user device;
obtaining, via an action plan engine, action plan data associated with the user profile;
analyzing, via a user data analysis engine, the user data to identify at least one of patterns, trends and shifts in the user data;
generating, via the user data analysis engine, feedback data to be provided to the at least one user device based on the analysis;
predicting, via the user data analysis engine, a crisis scenario based on the analysis; and
initiating, via a crisis intervention engine, a crisis intervention protocol when the crisis scenario is predicted, the crisis intervention protocol determined based on the assigned risk status.

12. The method of claim 11, wherein initiating the crisis intervention protocol comprises:
determining a communication protocol based on the assigned risk status;
initiating communication with the at least one user device according to the communication protocol;
activating a safety plan if a response is not received from the at least one user device within a predetermined time period.

13. The method of claim 12, wherein the communication protocol for a low risk status comprises at least one of generating a prompt via an application on the at least one user device and sending a text message to the at least one user device.

14. The method of claim 12, wherein the communication protocol for a high risk status comprises initiating a phone call to the at least one user device.
